(19) United States
(12) Patent Application Publication
(10) Pub. No.: US 2025/0171771 A1
(43) Pub. Date: May 29, 2025

(54) PROGRAMMING CELLULAR FUNCTION USING COMBINATORIAL GENETIC SCREENING

(71) Applicant: The Board of Trustees of the Leland Stanford Junior University, Stanford, CA (US)

(72) Inventors: Sandy L. KLEMM, Palo Alto, CA (US); Samuel Heeju KIM, Montrose, CA (US); Jacob A. BLUM, Redwood City, CA (US); William J. GREENLEAF, Menlo Park, CA (US)

(21) Appl. No.: 18/844,020

(22) PCT Filed: Mar. 16, 2023
(86) PCT No.: PCT/US2023/015413

§ 371 Date: Sep. 4, 2024

Related U.S. Application Data
(60) Provisional application No. 63/321,582, filed on Mar. 18, 2022.

(51) Int. Cl. C12N 15/10

(52) U.S. Cl. CPC C12N 15/1072; C12N 15/1082

(57) ABSTRACT

Described herein is a method for identifying combinations of perturbations that result in a cellular phenotype. In some embodiments, the method may comprise making a library of cells that have received combinations of perturbations, analyzing a subset of the cells at a single cell level, by measuring a phenotype in the cells and identifying which combinations of perturbations have been applied to the cells and, based on the results obtained from the analysis, calculating scores for the identified combinations of perturbations and scores for theoretical combinations of the perturbations, wherein each score indicates the likelihood that a combination of perturbation generates the phenotype.

### CROSS-REFERENCING

This application claims the benefit of U.S. provisional application Ser. No. 63/321,582, filed on Mar. 18, 2022, which application is incorporated by reference herein.

### GOVERNMENT SUPPORT

This invention was made with Government support under contracts HG007735 and HG009436 awarded by the National Institutes of Health. The Government has certain rights in the invention.

### BACKGROUND

Modern cellular therapies frequently use engineered (e.g., genetically modified) cells to perform specific tasks in patients. Clinical applications of these biologics demand complex phenotypes that often cannot be programmed into cells by modulating a single genetic pathway. Many biological processes in human cells are robust to perturbation of individual genes due to ubiquitous redundancy, and complex phenotypes often require synergistic activation of multiple genes. This intrinsic complexity of human cell biology presents a critical challenge to conventional, monogenic functional genomics that rely on single-gene perturbations. Consequently, there is a critical need to systematically identify combinations of genetic, epigenetic and pharmacological interventions that confer polygenic (involving multiple gene products) therapeutic functionality.

The key challenge in finding combinatorial solutions to cell engineering problems is that the scale of measurements required to make meaningful inference is intractable. For example, over a quadrillion independent measurements are required to naively phenotype all regulatory combinations within a universe consisting of as few as 50 potential regulators. Phenotyping even low complexity combinations, for example combinations with fewer than 5 components, would still require millions of experiments. Thus, there is a critical need for a scalable approach to combinatorial cell engineering.

### SUMMARY

Described herein is a method for identifying combinations of perturbations that result in a cellular phenotype. In some embodiments, the method may comprise making a library of cells that have received combinations of perturbations, analyzing a sub-set of the cells at a single cell level, by measuring a phenotype in the cells and identifying which combinations of perturbations have been applied to the cells and, based on the results obtained from the analysis, calculating scores for the identified combinations of perturbations (i.e., the combinations of perturbations identified in the cells) as well as theoretical combinations of the perturbations (i.e., combinations of the perturbations that are not identified in the cells), wherein each score indicates the likelihood that a combination of perturbation generates the phenotype. FIG. 1 illustrates some of the principles of this method.

In some embodiments the method may be iterative in the sense that the method may be performed and then repeated one or more times wherein, in each repeat, the library of cells is altered according to the calculated scores. For example, a repeat may be more focused on the combinations of perturbations that are more likely to generate the phenotype.

As illustrated in FIG. 1 and as will be explained in greater detail below, only a limited number of combinations of perturbations will be represented in the cells that are analyzed. However, based on the data obtained from those cells, scores for theoretical combinations of perturbations (i.e., combinations of perturbations that were not identified in the cells) can be calculated by learning algorithms. For example, in some embodiments, all possible pairwise, tri-wise, quad-wise, etc., up to n-wise combinations, where n is 5, 6, 7, 8, 9 or 10, up to the total number of perturbations) can be scored for their likelihood of causing the phenotype, where such combinations include theoretical combinations of the perturbations, i.e., combinations were not identified in the analyzed cells. The combinations of perturbations that are scored in the latter step of the method may include: i. the “observed” combinations of perturbations (i.e., the combinations of perturbations that were identified in the cells) and ii. the “theoretical” combinations of perturbations (i.e., the combinations of the perturbations that are not identified in the cells) where a theoretical combination can be; i. a new combination of the perturbations that is not in any of the analyzed cells or ii. a sub-combination of a combination of perturbations that has been identified in the analyzed cells. The scores for these combinations of perturbations (including the theoretical combinations) can be generated by statistical analysis of the collective data obtained from the cells, particularly by methods that employ learning algorithms. In any embodiment, likelihood scores for all potential combinations of the perturbations can be calculated.

This method, which may be referred to as “Combinatorial Cellular Programming” (CCP) below, provides the ability to systematically program biological cells with phenotypes that require manipulation of multiple genetic components. Certain principles of the method may be illustrated using the following hypothetical example. It is now known that reprogramming somatic cells into a pluripotent state requires the simultaneous exogenous expression of four transcription factors (Oct3/4, Sox2, Klf4, c-Myc) that are referred to as Yamanaka factors. Without any prior knowledge, associating these four transcription factors with a reprogramming phenotype would require testing an immense number of different combinations. In this example, even if one knew that four transcription factors were required, one would have to collect data from approximately 2×10¹¹ combinations if one wished to test all quad-wise combinations of the 1500 transcription factors encoded by the human genome. This is not physically possible. This phenotypic sparsity problem—where the correct solution makes up a small fraction of the total possible combinations—can be overcome by increasing the number of perturbation combinations that are being tested in each individual experiment. This solves the sparsity problem by increasing the frequency of “correct” solutions, but makes it more difficult to infer what the causative perturbation set is within each observed correct solution, as there are many perturbations that have no effect on the phenotype.

The premise of the present approach is that many important clinical phenotypes are regulated combinatorially and are not robustly accessible using single-gene perturbations. Combinatorial screening, however, introduces a seemingly intractable scaling problem: picking the right combination of genes to manipulate is impossible given the large numbers of possible combinations. Depending on how the method is performed, the present method leverages recent advances in machine learning, modern genome editing, and high-throughput single-cell phenotyping to resolve this combinatorial scaling problem, efficiently identifying combinations of genetic interventions that confer durable therapeutic function. One technological insight of the current approach is that an intractable, experimental problem of combinatorial cell engineering can be transformed into a scalable computational problem. This is achieved by constructing genetic perturbation libraries for which a combinatorial number of phenotypes can be extracted from each single cell.

Potential regulators are either up- or down-regulated at a multiplicity of perturbation (MoP, average number of perturbations per cell) above 1. This facilitates many combinations of perturbations to be analyzed in each cell (experimental compression). Individual cells are then phenotyped to provide paired perturbation and phenotype data to an inference (decompression) engine that identifies the causal regulators. The present platform should identify a new class of polygenic cellular therapeutics, not by sequentially modulating individual genes, but through an efficient, data-directed exploration of high-dimensional combinatorial perturbations. This approach enables phenotypic screening of trillions of combinatorial perturbations, revealing complex phenotypes that are unobservable through any monogenic screening approach. Together these innovations constitute a significant improvement in the art.

A split-pool method for exposing cells to perturbations is also provided. In these embodiments, partitioning cells into multiple partitions, selecting a sub-set of perturbations, applying subcombinations of the sub-set of perturbations to the partitions, optionally applying all of the perturbations in the sub-set to at least one of the partitions, optionally applying none of the perturbations in the sub-set to at least one of the partitions, pooling the cells, and repeating the method one or more times, wherein each repeat is done using a different sub-set of the perturbations. Details of this method are described in greater detail below.

These and other advantages may become apparent in view of the detailed description that follows below.

### BRIEF DESCRIPTION OF THE DRAWINGS

FIG. 1 is a flow chart illustrating some principles of the present method. In this example, a likelihood score is calculated is for all possible combinations of perturbations, including the theoretical combinations of perturbations that were not identified in the analyzed cells.

FIG. 2 is a flow chart illustrating an implementation of the present method. In this example, a likelihood score is calculated is for all possible combinations of perturbations, including the theoretical combinations of perturbations that were not identified in the analyzed cells.

FIG. 3 illustrates a combinatorial genetic screening workflow showing how causal factors of a phenotype are identified from a universe of potential regulators. Cells are loaded with combinatorial perturbations (Domain 1+2) and enriched for specific phenotypes (Domain 3). Positively selected cells are then genotyped to identify phenotypically causative perturbations (Domain 4). Finally, a new structured perturbation library is constructed (Domain 5) based on the information acquired during causal inference.

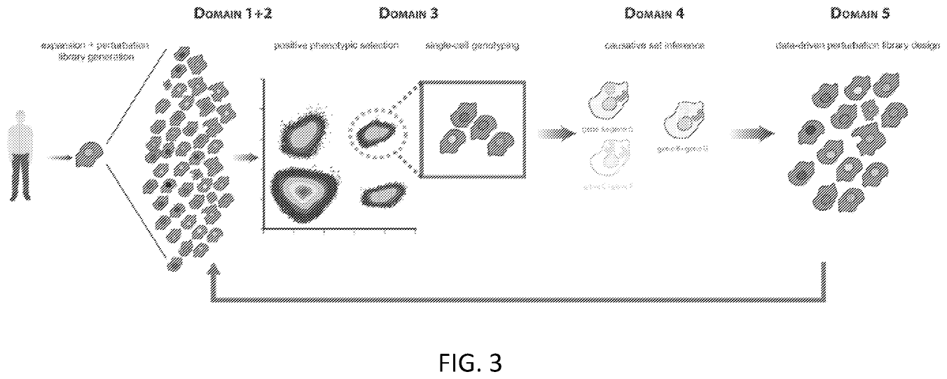

FIG. 4 illustrates how rare combinatorial solutions for complex phenotypes can be observed more frequently by introducing a high multiplicity of perturbation (MOP) per cell. Green denotes cells that have experienced a critical set of perturbations (with cardinality n) necessary to generate a specific phenotype. These cells may be exposed to other perturbations as well, which may negatively impact the phenotype of interest. However, for many applications of this technology, we are searching for a small number of causal perturbations within a large universe of unrelated perturbations. The number of observations per cell that match the required phenotypic complexity (n) are shown for various MOP levels (shaded blue). The observation frequency for a given phenotype is also reported for each MOP regime (shaded green).

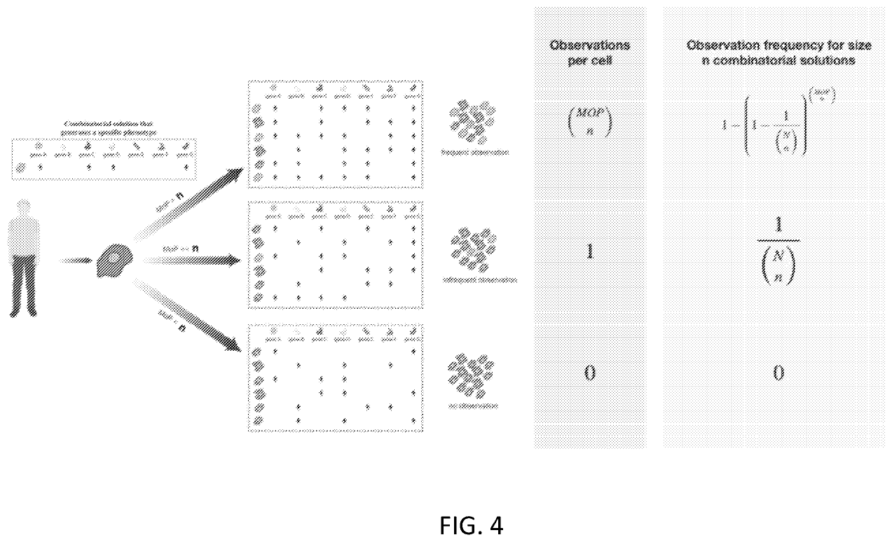

FIG. 5 illustrates an approach for constructing a combinatorial perturbation library using a split-and-pool method. In this method, each perturbation in the perturbation universe U is assigned to at least one of Q groups {P₁,P₂, . . . ,P_{Q}}. These assignments are either random, guided by prior biological knowledge (e.g., known synergistic or redundant relationships between epigenetic or genetic factors that are being perturbed), or designed using the active learning approach in the disclosed method. Next, progenitor cells for the library are split into K wells: (1) no perturbations are applied to the first well, (2) all perturbations in P₁ are applied to the second well, and (3) perturbation combinations {S₁,S₂, . . . ,S_{K-2}} drawn from P₁ (Sₖ is a subset of P₁ for each k) are applied to the remain K-2 wells. Finally, all cells are pooled into a single well, then re-split in K wells, repeating the same procedure for groups P₂ through P_{Q}. The distribution of perturbations for a hypothetical group P₂ is shown where the proteins represent perturbations. This procedure generates a perturbation library with complexity (number of different perturbation combinations) K^{Q}.

FIG. 6 illustrates how T Cell Receptor (TCR) complex can be displayed on the surface of a non-immune cell.

FIG. 7 illustrates a proof-of-principle probabilistic inference of causal components required for TCR display. Posterior probability of TCR display reported for all models with complexity 12 or lower (194,129,627 models shown). The model that is composed of the actual TCR components is indicated by a black square box.

### DEFINITIONS

As used herein, the term “perturbation” refers to any type of cellular manipulation, including but not limited to, introduction of constructs for the purposes of expressing or repressing a synthetic or endogenous gene product; or exogenous exposure of a cell to a drug, antibody, small molecule, or protein; or stimulation by physical force, including electromagnetic, temperature, pH, salinity or other non-molecular insult.

As used herein, the phrase “combinatorial perturbation” refers to a set of perturbations that are applied a cell.

As used herein, the phrase “perturbation library” refers to a collection of cells, each of which has been exposed to a set of perturbations or, equivalently, a combinatorial perturbation.

As used herein, the phrase “combinatorial perturbation library” refers to a “perturbation library” for which a subset of the constituent cells have more than one perturbation applied to them.

As used herein, the term “perturbation universe” refers to total set of perturbations that are possible or relevant for a particular cellular phenotype of interest.

As used herein, the phrase “multiplicity of perturbation” (herein also “MOP”) refers to the number of perturbations applied to a cell.

As used herein, the phrase “phenotypic complexity” or “complexity of the phenotype” refers to the minimal number of perturbations required to generate a given cellular phenotype.

As used herein, the phrase “causal perturbation” refers to the set of perturbations that is causally responsible for generating a given cellular phenotype.

As used herein, the phrase “high MOP” refers to a MOP that is higher than the phenotypic complexity

As used herein, the phrase “low MOP” refers to a MOP that is lower than the phenotypic complexity

As used herein, the phrase “unstructured perturbation library” refers to a perturbation library for which each cell is randomly assigned a set of perturbations

As used herein, the phrase “structured perturbation library” refers to a perturbation library for which each cell is assigned a non-random set of perturbations.

As used herein, the phrase “combinatorial assignment” refers to the assignment of perturbations to be applied to cells in a perturbation library according to the scheme outlined below.

As used herein, the phrase “active learning” refers to the process of using previously collected data to identify the most informative, unobserved, perturbation combinations to phenotype.

As used herein, the phrase “CRISPR machinery” refers to the collection of technologies that utilize CRISPR nucleoprotein complexes to regulate endogenous gene expression levels within a cell, including but not limited to CRISPR-Cas9 editing, CRISPR-interference, CRISPR-activation, CRISPR direct nucleoprotein delivery, CRISPR-Cas13 editing.

As used herein, the phrase “single-cell assay” refers to the collection of technologies that enable ensemble measurement of molecules in individual cells or cellular compartments, including but not limited to single-cell RNA-seq, single-cell ATAC-seq, single-cell CITE-seq, spatial transcriptomics, spatial metabolomics.

Before the present invention is further described, it is to be understood that this invention is not limited to embodiments described, as such may, of course, vary. It is also to be understood that the terminology used herein is for the purpose of describing certain embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a circuit” includes a plurality of such circuits and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

Certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

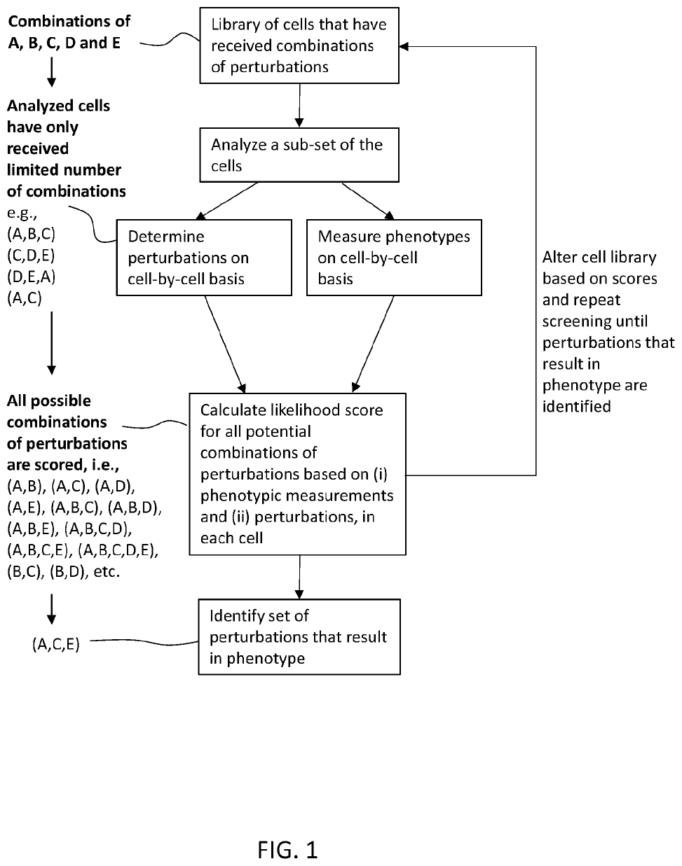

This disclosure provides, among other things, a method for identifying combinations of perturbations that result in a cellular phenotype. Certain principles of the method are illustrated in FIG. 1.

With reference to FIG. 1, in some embodiments, the method may comprise making a library of cells that have received combinations of perturbations. This library may be referred to as a “perturbation library” herein. The collective number of perturbations that have been received by the cells may be in the range of 10-5,000 or 20-1,000, for example. The average number of perturbations received by a cell may be at least 1, at least 2, at least 3, at least 4, at least 5, at least 10, at least 50 or at least 100, e.g., in the range of 5 to 10,000, 5 to 1000 or 5 to 500.

In the next step of the method illustrated in FIG. 1, a sub-set of the cells of the library is analyzed on a cell-by-cell basis. As shown, the cells are analyzed by (i) measuring a phenotype at a single cell level and (ii) at a single cell level, identifying which combinations of perturbations have been applied to the cells. These same cells are analyzed in this step, meaning that the perturbations that have been applied and the phenotypic measurements are determined for single cells. As shown, the cells that are analyzed have only received limited number of the possible combinations of perturbations (i.e., a relatively small subset of the “universe” of possible perturbations).

A phenotype may be measured using any suitable single-cell analysis method, e.g., by analyzing DNA, RNA, protein, and/or epigenetic modifications on a single cell basis. In these embodiments, the term “measured” is intended to mean a quantitative or qualitative assessment. In some embodiments, a phenotype may be measured by performing a single cell “omics” assay. Such assays may include “omics” and ‘multiomics” methods including, but not limited to RNA-seq (i.e., scRNA-seq), ATAC-seq (Assay for Transposase-Accessible Chromatin using sequencing, or csATAC-seq), CITE-seq (Cellular Indexing of Transcriptomes and Epitopes by Sequencing), scG&T-seq (single cell Genome & Transcriptome sequencing), scMT-seq (single cell Methylome and Transcriptome sequencing), scM&T-seq (single cell Methylome & Transcriptome sequencing), scTrio-seq (single-cell triple omics sequencing), scCOOL-seq (single cell Chromatin Overall Omic-scale Landscape Sequencing) and DOGMA-seq, among many others (see, generally, Islam et al (Genome Research. 2011 21: 1160-7), Valihrach et al (International Journal of Molecular Sciences 2018 19: 807), Zhu et al (Nat Methods. 2020 17:11-14) and Bode et al (Front Immunol. 2021 12: 702636), among many others). In some embodiments, the method may be done by detecting and/or measuring specific markers of the phenotype (e.g., the expression of cell surface markers, etc.) by FACS. Spatial assays may also be used in some cases. As may be apparent, this method may involve quantifying how similar a cell is to a cell that has a desired phenotype. In some embodiments, the phenotype of a cell may be measured while it is being enriched (e.g., by FACS). Identifying the specific set of perturbations present in a phenotyped cell may involve a direct, single-cell measurement of the genetic material mediating the perturbations (e.g., plasmid DNA or mRNA). Alternatively, this information may be acquired by single-cell sequencing an independent barcode that encodes the specific set of perturbation in a cell. These barcodes may be either transiently or permanently delivered by any convenient method.

In this method, the desired phenotype may have been characterized to some extent by prior work. For example, prior work may have established that cells that have a particular phenotype may have a defined gene expression pattern. In these embodiments, “measuring a phenotype” may be relatively straightforward in some cases and may involve identifying or quantifying the expression of one or more markers of the phenotype. In other cases, “measuring a phenotype” may be more complex and may involve gathering a large amount of measurements for a cell (e.g., by determining the transcriptome via RNA-seq) and then figuring out how similar the measurements are (as a whole) to the same type of measurements from a cell that has the phenotype. By way of example, if the goal is to identify perturbations that convert a stem cell into a liver cell, then one might use RNA-seq to figure out if liver cell markers are expressed in the cell and/or how similar the transcriptome is to the transcriptome of a liver cell. Methods for cross-comparing single cell omics data are known and can be readily adapted herein if desired (see, e.g., Alam et al Nat Genet 2021 53:1275), Adbadaal et al (Genome Biology 2019 volume 20: 194), Zhao et al (Proc Natl Acad Sci USA 2021 118:e2100293118) and Li et al (Front Immunol. 2021 Feb. 24; 12:625881) among many others).

It should be noted that in some embodiments, the phenotype may be measured and the perturbations may be identified in the same assay. For example, if RNA-seq is used (or a multi-omics method that includes RNA-seq) then the same data may be processed to identify the perturbations in that cell and measure the phenotype. In these embodiments the perturbations in a cell can be directly linked to the data obtained from that cell, which can make the statistical analysis steps of the method more accurate. Thus, in some embodiments, the phenotype may be measured and the combinations of perturbations have been applied to the cells are determined in the same analysis.

As shown in FIG. 1, the next step of the method may involve calculating scores for theoretical combinations of perturbations that were not identified in the analyzed cells, wherein each score indicates the likelihood that a combination of perturbation generates the phenotype. For clarity, a “theoretical” combination of perturbation contains perturbations that are not found together in the same cell (i.e., perturbations that are only found in different cells) as well as perturbations that are only present with other perturbations (i.e., as a “subcombination” of the perturbations identified in a cell). By way of example, the combination (A,B) would be considered a theoretical combination of perturbations if A and B are always in different cells. Likewise, the combination (A,B) would be considered a theoretical combination of perturbations if A and B are only found in a cell with another perturbation, e.g., C. These calculations are based on the results obtained from analysis step and, as such, are based on both of (i) the measurements of the phenotypic in each of the cells and (ii) the perturbations to which those cells have been exposed. In any embodiment and as illustrated in FIG. 1, this step may be done by calculating a score indicating the likelihood that each possible combination of the perturbations, i.e., the “universe” of possible combinations of perturbations, generates the phenotype. The “universe” of possible perturbations includes the theoretical combinations of perturbations (i.e., combinations that were not found in the cells). Algorithms for performing these calculations are described below.

Illustrated by a hypothetical example shown on the left side of FIG. 1 that that is solely being used to illustrate the principle of the method, if there are five perturbations: A, B, C, D and E and only four combinations of perturbations have been identified in the cells: (A,B,C), (C,D,E), (D,E,A) and (A,C), then up to 120 possible combinations of the perturbations, e.g., (A,B), (A,C), (A,D), (A,E), (A,B,C), (A,B,D), (A,B,E), (A,B,C,D), (A,B,C,E), (A,B,C,D,E), (B,C), (B,D) and so on may be scored. As illustrated, the scored combinations contain many theoretical combinations. In practice the number of theoretical perturbations that are given score may be much larger, and the combinations of perturbations in those cells will be more complex and numerous.

In some embodiments, the number of combinations of perturbations that are scored in this method may be at least 1 M, at least 10 M, at least 100 M, at least 1B, at least 10B, at least 100B or at least 1 T, depending on the collective number of perturbations that are analyzed at the beginning of the method. The difference between the number of combinations of perturbations that are identified in the cells and the number of combinations of perturbations that are scored may be large. For example, the number of combinations that are scored in this step may be at least 10 times, at least 100 times, at least 1000 times, at least 10,000 times, at least 100,000 times or at least 1 M times more than the number of combinations of perturbations that are identified in the cells. In some embodiments, at least all pairwise, tri-wise, quad-wise, 5-wise, 6-wise, etc., combinations, up to n-wise combinations, where n is up to 7, 8, 9, 10, or 20, for example, are scored. In some embodiments, the score is calculated using results obtained from cells that are positive for phenotype and results from cells that are negative for the phenotype. Details of the scoring algorithm may be found below. As would be apparent, scores for the combinations of perturbations that are found in the cells may be calculated at the same time. As such, in any embodiment, the method may involve calculating scores for the combinations of perturbations that were identified in the cells as well as calculating scores for theoretical combinations of perturbations that were not identified in the cells, i.e., the “theoretical” combinations described above. In some embodiments, all possible combinations are scored, including the combinations found in the cells and theoretical combinations that are not found in the cells.

In these embodiments, the term “score” is intended to refer to a number, letter, word (e.g., “high”, “medium” or “low”) or descriptor (e.g., “+++” or “++”) that can indicate the strength of the evidence that each potential combination of the perturbations causes the phenotype. A value can contain one component (e.g., a single number) or more than one component, depending on how a value is analyzed. In some embodiments, a score may be expressed as or based on a likelihood, probability or some other number that may be calculated using an algorithm.

The sub-set of cells analyzed may comprises one or more populations of enriched cells. In some embodiments, two distinct populations of enriched cells are analyzed: phenotypically positive cells and phenotypically negative cells, wherein the enriching is done by any convenient method, e.g., by cell sorting (FACS), enrichment on a support (e.g., bead enrichment), or a cell selection assay. For example, in some embodiments, cells may be enriched from the library by their expression of one or more cell surface markers that are associated with the phenotype, and the phenotype may be measured in those cells. In other embodiments, the sub-set of cells analyzed may comprise cells that are randomly sampled from the library. Regardless of the sub-sets of cells are produced, this step of the method will generally involve analyzing at least 1,000, at least 10,000, at least 100,000, at least 1 M or at least 10 M cells.

As indicated in FIG. 1, some embodiments may optionally comprise repeating the method one or more times (e.g., 2 or more, 5 or more, or 10 or more times), wherein in each repeat the sets of the perturbations that are applied to the cells at the beginning of the method are altered according to the scores calculated in the prior run. For example, at least one perturbation may be completely eliminated from the next round because it has a low likelihood of causing the phenotype and/or some combinations of perturbations may be prioritized. In the combinations are pre-determined, then if a pair or triplex of perturbations is calculated as having a relatively high likelihood of causing the phenotype (relative to other combinations) then it may be placed in two or more distinct sets of perturbations or placed in a set that has fewer additional perturbations, or in a set on its their own. As would be apparent, this step may require ranking the scores and/or applying a threshold to the scores to select the “best” combinations.

As indicated in FIG. 1, the method results in the identification of a minimal number of combination of perturbations that can generate the phenotype.

The perturbation library may be made in a variety of different ways. For example, the perturbation library may contain random combinations of perturbations. In these embodiments, the cells may be exposed to the perturbations en masse such that the cells are exposed to random combinations of the perturbations, for example. In another embodiment, the perturbation library may be made by partitioning cells into multiple partitions (e.g., at least 4, at least 8, at least 16 or at least 20 partitions), introducing various subsets of the perturbations to the partitions en masse and then pooling the cells. In this embodiment, the cells in each partition are exposed to random combinations of the perturbations that are added to that partition and then pooled.

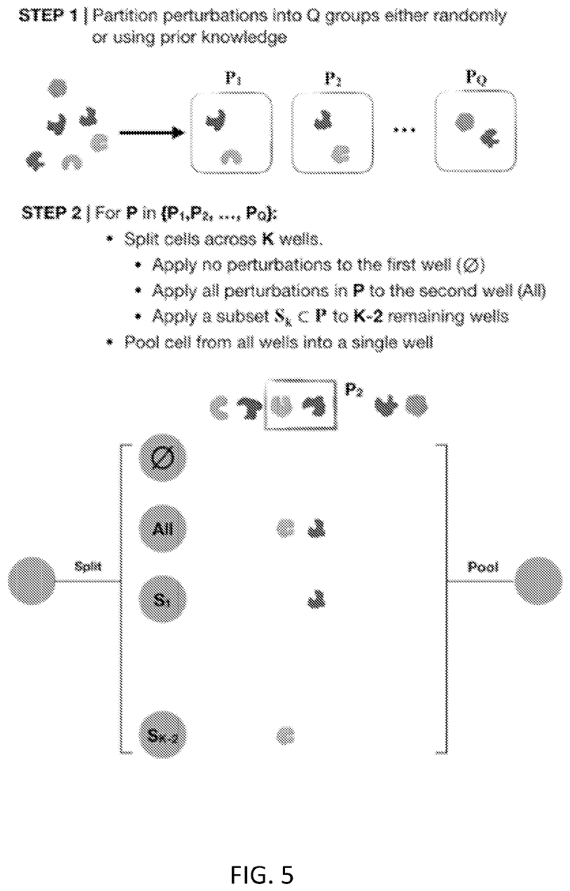

In other embodiments the perturbation library may contain pre-determined (i.e., not random) combinations of perturbations. These embodiments may be implemented using the “split-and-pool” method illustrated in FIG. 5, for example. This implemented of the method has advantages since the combinations of perturbations that are applied to the cells may be designed to maximize the efficiency of the discovery process. For example, if one combination of perturbations is calculated as being likely to cause the phenotype, then the library can be designed so that that particular combination is in more cells (potentially along with other perturbations).

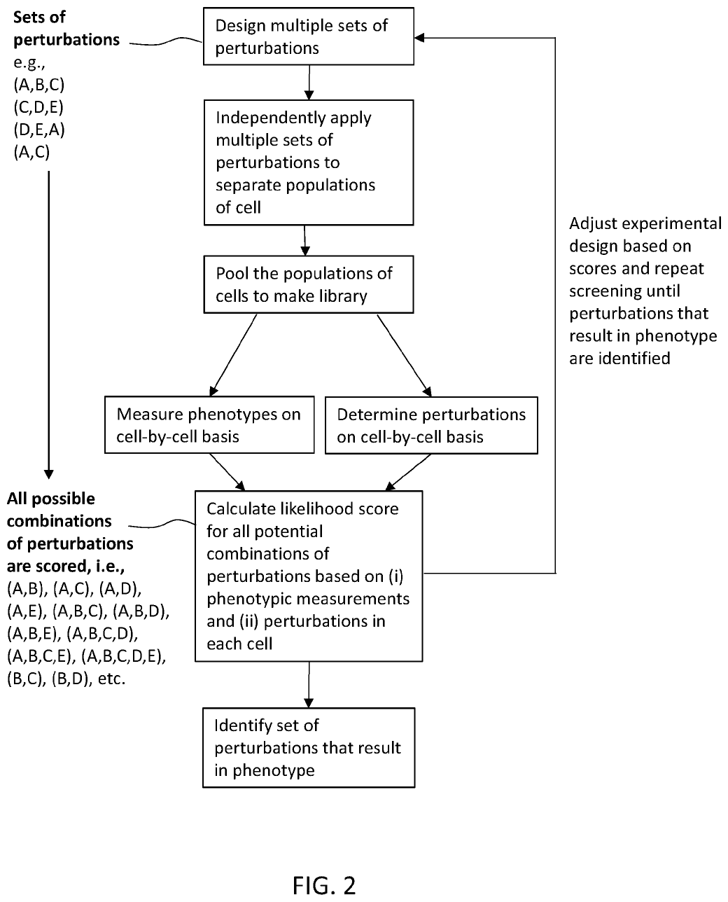

FIG. 2 is a flow chart illustrating an implementation of the method in which the perturbation library contains pre-determined combinations of perturbations (referred to as “sets of perturbations” in this figure). As shown in this figure, the sets of perturbations can be designed prior to being introduced to the cells. The remainder of the method is similar to that described above, except that the calculated scores alter the sets of perturbations that are applied to the cells. This step may be implemented using the split-and-pool based method described below.

As with the method shown in FIG. 1, the collective number of perturbations in the sets of FIG. 2 may be in the range of 10-5,000 or 20-1,000, for example. The average number of perturbations in a set may be at least 5, at least 10, at least 50 or at least 100, e.g., 5 to 10,000, 5 to 1000 or 5 to 500.

In any embodiment, the sets of perturbations may be applied to the cells using a split-and-pool approach. Split-and-pool based methods have generally been used for combinatorial chemistry and to index samples (see, e.g., Kuchina et al (Science 2021 371:eaba5257), O'Huallachain et al (Commun. Biol. 2020 3: 279), Cao et al (Science 2017 357: 661-667) and Rosenberg (Science 2018 360: 176-182)), among many others, not to introduce combinations of perturbations into cells. These publications are incorporated by reference for their descriptions of how split and pool methods can be implemented.

The principles of the present split-and-pool are illustrated in FIG. 5. This method may comprise: partitioning cells into multiple partitions (e.g., at least 4, at least 8, at least 16, at least 24 partitions at least 48 partitions or at least 96 partitions), selecting a sub-set of the perturbations, where the sub-set may contain 1 or more, 2 or more, 3 or more, 4 or more or 5 or more perturbations; applying subcombinations of the sub-set of perturbations to the partitions, pooling the cells, and then repeating the same steps one or more times (e.g., at least 2. at least 4, at least 10 or at least 20 times), wherein each repeat is done using a different sub-set of the perturbations. In some embodiments, the sub-set of perturbations that are selected in the initial round overlaps with at least one of the sub-sets of perturbations selected in a repeat. In some embodiments, the sub-set of perturbations that are selected in the initial round may not overlap with any of the sub-sets of perturbations that are selected in a repeat. In many embodiments, up to half, up to 75% or up to up to 90% of the partitions will receive a sub-set of perturbations in each round.

The sets of perturbations used in the method may be overlapping in the sense that in any single experiment one or more perturbations in one set may also be in another set. The sets may have the following characteristics in some cases: i. at least some of the sets of perturbations comprise multiple perturbations, ii. at least some of the perturbations are in more than one set, iii. at least one of the sets contains some but not all of the perturbations in another set and iv. collectively, the sets do not contain all potential combinations of the perturbations.

Illustrated by example, if there are 26 perturbations (A-Z) then the first sub-set of the perturbations may contain perturbations A, B, C and D and the subcombinations of the sub-sets of perturbations applied to the partitions applied to the partitions may include (A,B), (B,C), (C), (A,C,D) and, optionally (A,B,C,D). In a repeat: i. the sub-set of the perturbations may include perturbations D, E, F and G, and the sub-sets of the perturbations applied to the partitions applied to the partitions may include (D,E), (F), (E,F,G) and, optionally (D,E,F,G) if the subsets are overlapping (where D is the overlap), or the sub-set of the perturbations may include perturbations E, F, G and H, and the sub-sets of the perturbations applied to the partitions may include (E,F), (G), (E,G,H) and optionally (E,F,G,H) if the subsets are not overlapping.

In these embodiments, each population of cells in the partitions may have at least 100, at least 500, at least 1,000, at least 5,000 or at least 10,000 members, and the total number of cells in the pool will be over IM, e.g., at least 10 M.

As would be apparent, the perturbations are not chemically linked to each other in the method and there are no chemical addition or deprotection steps.

In some embodiments, the perturbations are nucleic acid constructs, wherein each construct encodes a perturbation. For example, the constructs may encode proteins, RNAs, or any combination thereof. In some embodiments, the perturbations are applied to the cells by introducing nucleic acid constructs into the cells, wherein each construct encodes a perturbation and multiple constructs are introduced into the cells, in a random or pre-determined way. In these embodiments, the constructs can encode protein (e.g., signaling proteins, transcription factors, enzymes, or protein fragments, etc.), RNA (e.g., a guide RNA, siRNA, aptamer, ribozyme, etc.), or any combination thereof (e.g., guide RNAs and an RNA guided protein such as an RNA-guided endonuclease, etc.), where the term “guide RNA” is intended to refer to an RNA that forms a complex with an RNA-guided protein (e.g., an protein such as AGO2, Cas9, Can13, Cas7-11, Cascade, Cpf1, Cas12, etc., including variants and fusion proteins thereof that have an additional enzymatic activity) and guides the protein to which it is complexed to a particular site or sequence in a nucleic acid (typically a sequence in the nuclear genome). For example, in some embodiments, the nucleic acid constructs may encode an open reading frame library (an “ORF library”) where the open reading frames may encode whole proteins, protein fragments, variants of a wild type protein, or proteins from another species, etc. A typical library will contain 10-5,000 or 20-1,000, constructs, for example.

In embodiments that use a guide RNA, the perturbation could result in a genetic alteration, e.g., a gene knockout. In other embodiments, the RNA-guided protein could be fused to methylase or demethylase. In these embodiments, the perturbation could result in a change in a methylation pattern. In other embodiments, the perturbation may the expression of a protein or an RNA. The constructs may be introduced into the cells by any convenient method, e.g., by lipid nanoparticles, viral transduction, transfection or electroporation. The perturbations may introduce permanent alterations to the cell through genomic integration—e.g. viral or transposon-based (e.g., PiggyfBac) delivery—or transient effects (e.g., plasmid, dsDNA or RNA electroporation).

In other embodiments, the perturbations may be non-nucleic acid molecules, e.g., a drug, antibody, small molecule, or protein or a stimulus such as a physical stimulus, e.g., including electromagnetic, temperature, pH, salinity or other non-molecular insult. In these embodiments, the partitioned cells may be barcoded in step (b), wherein the barcode indicates which perturbation has been applied to the cells. This barcode could be, for example, on a construct that is added to the cells at the same time as the perturbation. In these embodiments the construct may be non-functional in the sense that it does not actually encode the perturbation. However, it identifies the perturbation that was added at the same time. As such, as cells accumulate perturbations, they should accumulate the barcodes that encode those perturbations.

Any implementation of the method may use a combination of nucleic acid-based perturbations and non-nucleic acid-based perturbations.

Further details may be found in the following description.

### Phenotypes

The phenotype that is measured may be molecular (e.g., the levels or positioning of cell surface proteins, nuclear localized proteins (e.g., transcription factors), or cytoplasmic proteins (e.g., cytokines)) or functional. In the latter case, phagocytosis, tissue- or signal-specific cellular localization could be measured. In some implementations, a library of perturbed cells can be introduced into an organism (e.g., a mouse, monkey or human) and then extracted for molecular, functional and/or localization phenotyping. In these embodiments, a sample from the organism may be tested too. In some embodiments, the phenotyping can be performed hierarchically. For example, a sub-library of cells may be selected by high-throughput molecular phenotyping (e.g., surface protein expression) and subsequently used as the input library for lower-throughput molecular (e.g., whole transcriptome single-cell RNA-seq) or functional (in-vitro or in-vivo) phenotyping.

### Probabilistic Modeling

The posterior probability that a given perturbation combination (c) confers the phenotype of interest (“P(c)”) can be estimated by many statistical methods. In some embodiments, the posterior probability that a given perturbation combination confers the phenotype of interest will be estimated by training and applying an ensemble decision tree statistical model. In other embodiments, the posterior probability that a given perturbation combination confers the phenotype of interest will be estimated by training and applying a random forest statistical model. In yet other embodiments, the posterior probability that a given perturbation combination confers the phenotype of interest will be estimated by training and applying a neural network model. The posterior probability that a set of perturbations comprises the full complement of causal regulators can be calculated directly from P(c). See Appendix A, Section 0.1.

### Active Learning

Active learning as applied to this method relates to the use of information metrics to identify maximally informative perturbation combinations to phenotype. See Appendix A, Section 0.2 for an example of active learning as used by the disclosed method.

### Combinatorial Perturbation Library Construction

In embodiments that involve a structured perturbation library, one approach to building highly diverse, high MOP libraries, hereinafter referred to as “combinatorial perturbation assignment,” “combinatorial assignment,” “combinatorial library construction,” or “split-and-pool library constructions” is illustrated in FIG. 5. Briefly, each perturbation in the perturbation universe U is assigned to one of Q groups {P₁,P₂, . . . ,P_{Q}}. These assignments are either random, guided by prior biological knowledge (e.g., known synergistic or redundant relationships between epigenetic or genetic factors that are being perturbed), or designed using the active learning as outlined in the disclosed method. Next, progenitor cells for the library are split into K wells: (1) no perturbations are applied to the first well, (2) all perturbations in P₁ are applied to the second well, and (3) perturbation combinations {S₁,S₂, . . . ,S_{K-2}} each composed from P₁ (Sₖ is a subset of P₁ for each k) are applied individually to the remain K-2 wells. Finally, all cells are pooled into a single well, then re-split in K wells, repeating the same procedure for groups P₂ through P_{Q}.

This procedure generates a perturbation library with complexity (number of unique perturbation combinations) equal to K^{Q}.

For some embodiments of this procedure, the number of wells (K in this example) into which cells are split during each round will vary, depending on the composition of the perturbation group.

For some embodiments of this procedure, the no perturbation and/or all perturbation wells may be eliminated.

### EXAMPLES

For K=2, at each round of the split-and-pool procedure, every cell is either exposed to all perturbations or no perturbations from the relevant group.

A further case is realized when each perturbation subset Sₖ contains all but the k^{th} element of the group Pᵢ. In this case K=n+2, where n is the number of perturbations in each group.

In general, the subsets Sₖ will be designed by an active learning algorithm as described in this disclosure.

### Perturbation Libraries

Perturbation libraries may be unstructured or structured, the details of which can be found below.

For an unstructured perturbation library, in one embodiment the disclosure provides a method for identifying sets of perturbations that confer a specified phenotype comprising:
applying a set of perturbations randomly selected from the perturbation universe to each cell in the perturbation library, wherein the average number of perturbations per cell (MOP) is greater than the phenotypic complexity;
identifying the specific perturbations applied to each cell with a set of cells that are positive for the phenotype, and identifying the specific perturbations applied to each cell within a set of cells that are negative for the phenotype (this may or may not involve physical separation a sets of cells that are positive and negative for the given phenotype;
calculating, from the data acquired in (2.2), for all possible perturbation combinations the probability that a set of perturbations comprises the full complement of causal regulators (See Appendix A, Section 0.1, Equation 1), and/or the probability that a set of perturbations confers the phenotype of interest, and/or a priority ranked list of perturbation combinations for subsequent analysis or experimentation.

For a structured perturbation library, in another embodiment the disclosure provides a method for identifying sets of perturbations that confer a specified phenotype comprising:
applying a set of perturbations assigned by combinatorial assignment (see Combinatorial perturbation library construction below), wherein the average number of perturbations per cell (MOP) is greater than the phenotypic complexity;
identifying the specific perturbations applied to each cell with a set of cells that are positive for the phenotype, and identifying the specific perturbations applied to each cell within a set of cells that are negative for the phenotype (this may or may not involve physical separation a sets of cells that are positive and negative for the given phenotype);
calculating, from the data acquired in (3.2), for all possible perturbation combinations the probability that a set of perturbations comprises the full complement of causal regulators (See Appendix A, Section 0.1, Equation 1), and/or the probability that a set of perturbations confers the phenotype of interest, and/or a priority ranked list of perturbation combinations for subsequent analysis or experimentation.

For a structured perturbation library with active learning in one embodiment, the disclosure provides a method for identifying sets of perturbations that confer a specified phenotype comprising:
applying a set of perturbations to each cell in the perturbation library, wherein the perturbations are drawn from the perturbation universe through either random selection or combinatorial assignment, and wherein the average number of perturbations per cell (MOP) is greater than the phenotypic complexity;
identifying the specific perturbations applied to each cell with a set of cells that are positive for the phenotype, and identifying the specific perturbations applied to each cell within a set of cells that are negative for the phenotype (this may or may not involve physical separation a sets of cells that are positive and negative for the given phenotype);
calculating, from the data acquired in (4.2), for all possible perturbation combinations the probability that a set of perturbations comprises the full complement of causal regulators, and/or the probability that a set of perturbations confers the phenotype of interest, and/or a priority ranked list of perturbation combinations for subsequent analysis or experimentation;
identifying a collection of maximally informative, unobserved, perturbation sets that can be applied combinatorially to cells as an input to 4.1, and proceeding iteratively as an active learning algorithm (See Appendix A, Section 0.2, Equation 3).

### Statistical Inference Methods

Examples of statistical inference methods are shown in Appendix A, shown below:
Let D={(vₙ, ω₀), . . . , (vₙ, ωₙ)} denote a set of data where pₖ is 1 if a perturbation set wₖ is observed within a cell positive for a given phenotype, and 0 otherwise. Let Pᵥ(ω) be the probability that a perturbation combination ω confers the phenotype v∈{0, 1}. The distribution Pᵥ(ω) is trained using the data D. If M is the set of all possible perturbation combinations, and Mₒ is the set of observed combinations, then for each ω∈M, the posterior probability that ω comprises all and only causal regulators of the phenotype is given by

$$P ( ω \vert D ) = \frac{{2}^{- n \vert ω \vert} {\prod }_{c ∈ ℳ} \text{?} {P}_{1} ( ω ⋂ c ) \text{?} {P}_{0} ( ω ⋂ c ) \text{?}}{{\sum }_{{\sum }_{σ ∈ ℳ} {2}^{- n [ σ ]}} {\prod }_{c ∈ ℳ} \text{?} {P}_{1} ( σ ⋂ c ) \text{?} {P}_{0} ( σ ⋂ c ) \text{?}} . \qquad ( 1 )$$

$$\text{?} \text{indicates text missing or illegible when filed}$$

where |·| is the cardinally operator, and n_{c}⁺ and n_{c}⁻ denote the number of times a given perturbation set c is observed in phenotypically positive and negative cells, respectively.

### 0.2 Active Learning: Phenotyping Maximally Informative Perturbation Combinations

The information I gained by phenotyping a perturbation set α∈M is given by

$$I [ σ ] = {H}_{ω} - {H}_{ω \vert σ} . \qquad ( 2 )$$

wherein D is the set of data collected at the point of selection. H_{ω} is the entropy of ω₁ and H_{ω/σ} is the conditional entropy of ω given σ. Intuitively I[σ] is the expected reduction in entropy of ω (or information gained) that would be realized if the phenotype of σ were known. By equation 2, we calculate the putative information gain from phenotyping σ as follows:

$$I [ σ ] = {\sum }_{ω ∈ ℳ} P ( ω \vert D ) {log}_{2} P ( ω \vert D ) - {\sum }_{ω ∈ ℳ} \text{?} {P}_{v} ( σ ) P ( ω \vert \{ v , σ \} ⋃ D ) {log}_{2} P ( ω \vert \{ v , σ \} ⋃ D ) , \qquad ( 3 )$$

$$\text{?} \text{indicates text missing or illegible when filed}$$

where ω|D˜P_{ω/D} is given in equation 1. In the proposed active learning framework, perturbation combinations are selected for phenotyping to maximize the information gain as calculated in equation 3.

### Cells

In any embodiment, the cells may be mammalian cells.

Suitable cells include stem cells, progenitor cells, as well as partially and fully differentiated cells. Suitable cells include, neurons, liver cells; kidney cells; immune cells; cardiac cells; skeletal muscle cells; smooth muscle cells; lung cells; and the like.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, autotransplated expanded cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogenic cells, allogenic cells, and post-natal stem cells.

In some cases, the cells are a stem cells. In some cases, the cell is an induced pluripotent stem cell. In some cases, the cell is a mesenchymal stem cell. In some cases, the cell is a hematopoietic stem cell. In some cases, the cell is an adult stem cell.

Suitable cells include bronchioalveolar stem cells (BASCs), bulge epithelial stem cells (bESCs), corneal epithelial stem cells (CESCs), cardiac stem cells (CSCs), epidermal neural crest stem cells (eNCSCs), embryonic stem cells (ESCs), endothelial progenitor cells (EPCs), hepatic oval cells (HOCs), hematopoetic stem cells (HSCs), keratinocyte stem cells (KSCs), mesenchymal stem cells (MSCs), neuronal stem cells (NSCs), pancreatic stem cells (PSCs), retinal stem cells (RSCs), and skin-derived precursors (SKPs).

In some instances, a cell is an immune cell. Suitable mammalian immune cells include primary cells and immortalized cell lines. Suitable mammalian cell lines include human cell lines, non-human primate cell lines, rodent (e.g., mouse, rat) cell lines, and the like. In some instances, the cell is not an immortalized cell line, but is instead a cell (e.g., a primary cell) obtained from an individual. For example, in some cases, the cell is an immune cell, immune cell progenitor or immune stem cell obtained from an individual. As an example, the cell is a lymphoid cell, e.g., a lymphocyte, or progenitor thereof, obtained from an individual. As another example, the cell is a cytotoxic cell, or progenitor thereof, obtained from an individual. As another example, the cell is a stem cell or progenitor cell obtained from an individual.

As used herein, the term “immune cells” generally includes white blood cells (leukocytes) which are derived from hematopoietic stem cells (HSC) produced in the bone marrow. “Immune cells” includes, e.g., lymphoid cells, i.e., lymphocytes (T cells, B cells, natural killer (NK) cells), and myeloid-derived cells (neutrophil, eosinophil, basophil, monocyte, macrophage, dendritic cells). “T cell” includes all types of immune cells expressing CD3 including T-helper cells (CD4+ cells), cytotoxic T-cells (CD8+ cells), T-regulatory cells (Treg) and gamma-delta T cells. A “cytotoxic cell” includes CD8+ T cells, natural-killer (NK) cells, and neutrophils, which cells are capable of mediating cytotoxicity responses. “B cell” includes mature and immature cells of the B cell lineage including e.g., cells that express CD19 such as Pre B cells, Immature B cells, Mature B cells, Memory B cells and plasmablasts. Immune cells also include B cell progenitors such as Pro B cells and B cell lineage derivatives such as plasma cells.

In other embodiments, the cell may be a cancer cell, e.g., a malignant cell that is grown in culture.

### Utility

The method finds use in identifying perturbations that can generate a particular phenotype. The method finds use to, e.g., identify perturbations that cause stem cells to differentiate in a particular way (e.g., to any of the cell types listed above), or to identify perturbations would make therapeutic cells more effective (e.g., to reduce T cell exhaustion), etc.

In particular, the method can be used to identify perturbations that cause cellular differentiation, reprogramming, and/or trans-differentiation. Examples of uses include identifying perturbations that can (1) differentiate induced pluripotent stem cells (iPSCs) into human cells with therapeutic or regenerative potential (e.g., cytotoxic or anti-inflammatory T cells), (2) regenerate a pool of non-renewing cells (e.g., neurons) from proximal, renewable populations (e.g., astrocytes, microglia) by transdifferentiation, (3) stabilize an existing cell type (e.g., exhaustion resistance for cytotoxic T cells or inflammation resistance for regulatory T cells), or (4) build a hybrid cell type that combines therapeutically advantageous properties from multiple human or non-human cell types. These and other utilities should be readily apparent.

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight and all solvent mixture proportions are by volume unless otherwise noted.

### EXAMPLES

### Example 1

### Combinatorial Identification of Genes that Confer T Cell Receptor (TCR) Cell Surface Display on a Non-Immune Cell

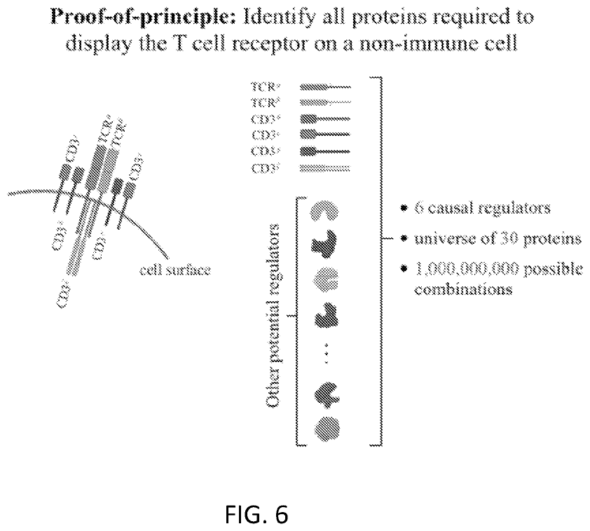

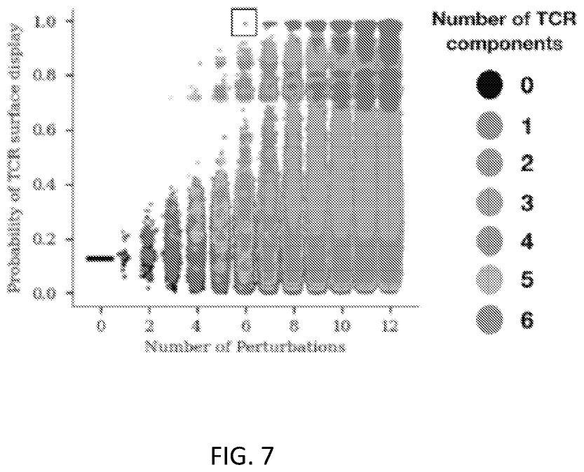

To illustrate the present method, the following proof-of-principle experiment was conceived: use the method to identify all molecular components of the T cell receptor complex that are required for cell surface display (FIG. 6). Six proteins are required to display the TCR complex on non-immune cells, and the task is to distinguish these proteins from 24 other unrelated factors. The universe of perturbations in this case is the set of 30 distinct genes that can be overexpressed in the target cells (6 TCR components and 24 unrelated factors). A perturbation library consisting of 86 perturbation types with an average MOP of 14 was constructed. TCR positive and negative cells were isolated by flow cytometry and subjected to single-cell RNA sequencing to identify the set of perturbation applied to each cell. All 86 perturbation types were identified with an average of 56 cells observed for each perturbation type. These data were then used to train a binary classifier using an ensemble (bagged) decision tree statistical model. This trained model was then used to estimate the posterior probability of TCR display for each unobserved combination of perturbations (1,073,741,824 models were analyzed). These estimates clearly show that the actual components of the TCR are the most likely predictors of TCR display relative to other models of similar complexity (FIG. 7).

We claim:

1. A method for identifying combinations of perturbations that result in a cellular phenotype, comprising:
(a) making a library of cells that have received combinations of perturbations;
(b) analyzing a sub-set of the cells at a single cell level, by:
(i) measuring a phenotype in the cells; and
(ii) identifying which combinations of perturbations have been applied to the cells;
(c) based on the results of (b), calculating scores for theoretical combinations of the perturbations that were not identified in (b)(ii), wherein each score indicates the likelihood that a combination of perturbations generates the phenotype; and
(d) optionally repeating step (a)-(c) one or more times, wherein in each repeat the library of cells of (a) is altered according to the scores calculated in step (c).

2. The method of claim 1, wherein on average the cells of (a) have each received least 5 perturbations.

3. The method of claim 1, wherein the sub-set of cells analyzed in (b) comprises one or more populations of enriched cells.

4. The method of claim 3, wherein the enriched cells comprise phenotypically positive and/or phenotypically negative cells.

5. The method of claim 3 or 4, wherein the enriching is done by cell sorting, bead capture, or a cell selection assay.

6. The method of claim 1, wherein the sub-set of cells analyzed in (b) comprises cells that are randomly sampled from the library of (a).

7. The method of claim 1, wherein (b)(i) and (b)(ii) are done in same analysis.

8. The method of claim 1, wherein the analysis includes analyzing DNA, RNA, protein, epigenetic modifications, metabolites and/or spatial distributions in single cells.

9. The method of any prior claim, wherein the collective total number of perturbations applied in step (a) is at least 20.

10. The method of any prior claim, wherein at least 1 M scores are calculated in step (c).

11. The method of any prior claim, wherein in step (a) the perturbations are applied to the cells by introducing nucleic acid constructs into the cells.

12. The method of claim 10, wherein the constructs encode protein, RNA, or any combination thereof.

13. The method of any of claims 1-11, wherein in step (a) the perturbations are applied to the cells by introducing a non-nucleic acid molecule or a physical stimulus to the cells.

14. The method of any prior claim, wherein step (a) is done using a split-and-pool approach, and the cells have received pre-determined sets of perturbations.

15. The method of any prior claim, wherein the score of (c) is calculated using data obtained from cells that have the phenotype and data obtained from cells that do not have the phenotype.

16. The method of any prior claim, wherein the cells are mammalian cells.

17. The method of any prior claim, wherein (c) comprises calculating scores for all potential combinations of the perturbations, wherein each score indicates the likelihood that a combination of perturbations generates the phenotype.

18. A split-and-pool method for exposing cells to perturbations, comprising:
(a) partitioning cells into multiple partitions;
(b) selecting a sub-set of perturbations;
(c) applying subcombinations of the sub-set of perturbations to the partitions;
(d) optionally applying all of the perturbations in the sub-set to a partition;
(e) optionally applying none of the perturbations to a partition;
(f) pooling the cells after (e); and
(g) repeating steps (a)-(f) one or more times, wherein each repeat is done using a different sub-set of the perturbations.

19. The method of claim 18, wherein the sub-set of perturbations that are selected in (b) overlaps with at least one of the sub-sets of perturbations selected in a repeat of (g).

20. The method of claim 18, wherein the sub-set of perturbations that are selected in (b) does not overlap with any of the sub-sets of perturbations that are selected in a repeat of (g)

21. The method of any of claims 18-20, wherein there is no chemical addition or deprotection step in the method.

22. The method of any of claims 18-21, wherein (g) comprises repeating steps (a)-(f) at least 2 times.

23. The method of any of claims 18-22, wherein (a) comprises partitioning the cells into at least 4 partitions.

24. The method of any of claims 18-23, wherein there are at least IM cells.

25. The method of any of claims 18-24, wherein the perturbations are nucleic acid constructs, wherein each construct encodes a perturbation.

26. The method of claim 25, wherein the construct encodes a protein, an RNA, or any combination thereof.

27. The method of any of claims 18-26, wherein the perturbations are small molecules, wherein each small molecule is a perturbation.

28. The method of any of claims 18-27, wherein the partitioned cells are barcoded in steps (c)-(d), wherein the barcode indicates which perturbation has been applied to the cells.

29. The method of any of claims 18-28, wherein the cells are mammalian cells.
